(19) United States
(12) Patent Application Publication
(10) Pub. No.: US 2005/0147525 A1
(43) Pub. Date: Jul. 7, 2005

(54) Sanitized tubing termination method and assembly

(76) Inventor: Gerald G. Bousquet, Tyngsborough, MA (US)

Correspondence Address: CESARI AND MCKENNA, LLP, 88 BLACK FALCON AVENUE, BOSTON, MA, 02210, US

(21) Appl. No.: 10/848,035

(22) Filed: May 18, 2004

Related U.S. Application Data
(63) Continuation-in-part of application No. 10/752,475, filed on Jan. 6, 2004.

(51) Int. Cl. A01N 1/00

(52) U.S. Cl. 422/28; 604/538

(57) ABSTRACT

A catheter termination assembly includes a catheter connection of the type having a coupling stem at one end, a catheter connection nipple at the opposite end, an axial lumen extending between those ends, and a transverse enlargement between the stem and the nipple. The assembly also includes cup-like end cap, the end cap having an end wall, and a skirt extending from the end wall. The skirt is sized to engage around the enlargement and interfitting surfaces on the enlargement and interior wall of the skirt enable the skirt to be releasably coupled to the enlargement so as to define a fluid-tight chamber encircling the stem. The end cap has a fluid injection site by which a sanitizing agent may be present in or introduced into the chamber so as to immerse the stem in the sterilizing agent. A method of sanitizing a tubing connector fitted with the end cap is also disclosed.

### RELATED APPLICATION

This application is a continuation-in-part of Ser. No. 10/752,475, filed Jan. 6, 2004, now ______.

### BACKGROUND OF THE INVENTION

This invention relates to a method and apparatus for inhibiting or eliminating the colonization by microorganisms of in-dwelling medical devices and the subsequent occurrence of infections associated with same.

1. Field of the Invention

Infections associated with medical care are a major cause of morbidity and mortality. These infections are typically very costly to manage and may be associated with a variety of adverse outcomes including death. Common types of infections that occur in an acute care setting include pneumonia, wound infection and blood stream infection. Often these infections develop from the use of invasive devices in patients with limited resistance to infection as a result of the underlying illness or drug therapy. Also, because many strains of microorganisms acquired in the hospital are resistant to commonly used antibiotics, it is often very difficult and costly to treat these infections.

Medical catheter or device-related infections such as those associated with intravenous, intraarterael, dialysis and other types of medical catheters or implanted medical devices usually result because of a breach of natural protective mechanisms present at sites such as the skin. In these situations, the development of infection is believed to involve the following steps:
1. Microorganisms colonize and multiply in a patient's skin. There is a microbial flora normally present on the skin. When a patient is admitted to a healthcare setting, organisms from this environment (typically resistant to antimicrobials) become part of the patient's flora soon after admission;
2. After insertion of the catheter or other medical device, these organisms continue to multiply and begin to colonize the surface of the catheter or device;
3. As the organisms continue to multiply, they spread on the external and/or internal surfaces of the catheter or device and eventually enter normally sterile tissues (e.g. blood vessels, abdomen, etc.). This process is facilitated by the development of a biofilm (which consists of proteins from the blood or tissue fluid and other debris) on the surface of the devices, and
4. Once in these normally sterile sites, the organisms continue to multiply and the development of a clinical infection may result.

It has been known for many years that staphylococcus aureus, for example, nearly always can be found to colonize the patients that develop infection with this organism, colonization meaning the presence of the organism without evidence of infection.

One of the most important sites for colonization is the anterior part of the nose (anterior nares). Individuals that develop a staphylococcal infection at the site of an indwelling catheter or device usually have nasal colonization with this organism.

2. Description of the Prior Art

One device category that is particularly susceptible to biological contamination are the fittings or connectors at the ends of catheters implanted in patients to provide prolonged or repeated access to the internal organs of those patients. For example, a central venous catheter (CVC) is used to access a patient's venous system for the administration of intravenous (IV) fluids, antibiotics and chemotherapy. Catheters are also implanted in patients who require repeated access to the peritoneum for hemodialysis peritoneal dialysis. Such a catheter exits the patient's body at the skin line usually through a standard transcutaneous access device. A short length of catheter resides outside the body and is terminated by an end fitting, often a male luer connector or the like. When treating the patient, that connector is coupled to a mating female connector at the end of a length of tubing leading to a dialysis machine, fluid administration set or the like.

When the therapy session is completed, the mated connectors are decoupled and the connector at the end of the catheter extending from the patient is closed by an end cap which is basically a blind or dead end version of the female connector. Invariably prior to affixing the end cap, the male connector is sanitized or disinfected by wiping it down with a disinfectant such as alcohol, bleach or betadyne. However, I have found through testing that even after such a wipedown, appreciable biological contamination still exists on the connector due to the fact that the connector has an exterior thread or crannies and crevasses which are difficult to reach with a disinfectant wipe.

Resultantly, when the connector is again coupled to the mating connector of the dialysis machine or administration set, biological contaminants may be entrained in the fluid flow to the patient giving rise to infection and possible sepsis.

A variety of techniques have been used in an attempt to reduce the frequency of the infections described above. These have included impregnating the catheter connectors with antibiotic or photodynamic substances, incorporating silver or silver compounds in the connectors and irradiating the devices with infrared or ultraviolet light. Each of these prior techniques has, to some extent, reduced the frequency of infection. However, none of them is ideal. An ongoing concern is that microorganisms have the potential to develop resistance to antimicrobials incorporated into the catheter or device in an attempt to prevent infection. Thus, there is an on-going need for an improved mechanism for sanitizing the end fittings of in-dwelling medical catheters.

### SUMMARY OF THE INVENTION

Accordingly, it is an object of the present invention to provide a method for sanitizing or disinfecting medical tubing connectors and terminations.

A further object is to provide a sanitized termination assembly for medical tubing.

Another object of the invention is to provide a sterile catheter connector terminations assembly.

Still another object is to provide an assembly such as this which is relatively easy and inexpensive to make in quantity.

Yet another object of the invention is to provide a device for maintaining the sterility of the connector of an in-dwelling medical catheter in a sanitized condition.

Other objects will, in part, be obvious and will, in part, appear hereinafter.

The invention accordingly comprises the features of construction, combination of elements and arrangement of parts which will be exemplified in the construction hereinafter set forth, and the scope of the invention will be indicated in the claims.

Briefly, my catheter or tubing termination assembly comprises a connector which, in use, is secured to the exposed end of a catheter or length of tubing implanted in a patient usually by way of a transcutaneous access device; see, e.g., my U.S. Pat. No. 5,662,616. The connector may have a more or less standard configuration, i.e. like that of a luer connector or screw connector commonly used in the medical industry. Often the connector that is attached to the catheter implanted in the patient is a male connector which is adapted to be coupled to a corresponding female connector having the same configuration. The present connector is more or less standard in that it has a tubular body with a coupling stem at one end, a catheter nipple at the other end and a lumen extending between the stem and nipple. The coupling stem is the part of the connector most prone to transferring biological contamination to the patient.

The connector of this invention may differ from a standard medical connector only in that its body may have an enlargement or flange between its ends and the periphery of that enlargement or flange is threaded or otherwise shaped to make a releasable connection with an end cap to be described now.

The other component of the termination assembly is a cup-like end cap which may be retrieved from a sterile package. The end cap has internal wall surfaces which interfit with surfaces of the connector enlargement or flange so that the end cap and connector may be coupled together to form a fluid tight enclosure or chamber containing the coupling stem of the connector.

In accordance with the invention, a sanitizing agent is present in, or may be introduced into the chamber, so as to immerse the connector stem in that agent for a long enough time to completely sanitize or disinfect that stem. The agent may be introduced at an injection site in a wall of the end cap. That site may comprise a needle-penetrable septum so that the agent may be injected into the chamber using a syringe with a hypodermic needle. Alternatively, that site may comprise a valved injection port for a so-called needleless syringe. The valve in such a port opens automatically when the syringe is connected to the port. In some applications, the disinfecting agent may be contained in an antechamber in the end cap itself and released when the end cap is coupled to the connector as will be described later.

In one embodiment of the invention, the sanitizing agent is a chemical disinfectant such as alcohol, bleach, betadyne or the like. Therefore, in order to prevent the agent from entering the lumen of the connector body, and thus gaining entry to the patent the end cap includes means for blocking the mouth of the connector stem lumen when the cap is coupled to the connector.

In other invention embodiments, such blocking is not required because the chamber surrounding the connector stem is filled with a biocompatible sanitizing agent such as saline solution which would not harm the patient even if it did enter the lumen. The volume of solution in that chamber may then be heated by microwave energy or by other means to a temperature sufficient to kill any microbials resident on the connector stem.

In all invention embodiments, the end cap and its now sanitized contents may remain on the connector until it is necessary to couple that connector to a mating connector at the end of a catheter leading to a dialysis machine, administration set or other therapeutic apparatus. When that time arrives, the termination may be pointed downward and the end cap released from the connector such that the disinfecting agent remains in the end cap. The end cap may then be thrown away or sterilized for reuse. The sanitized connector stem may then be coupled to its mate in the usual way with assurance that there has been no bacterial formation on the connector stem during its period of nonuse.

### BRIEF DESCRIPTION OF THE DRAWINGS

For a fuller understanding of the nature and objects of the invention, reference should be made to the following detailed description, taken in connection with the accompanying drawings, in which:

FIG. 1 is a diagrammatic view showing a pair of in-dwelling catheters which are terminated according to the invention;

FIG. 2A is a side elevational view with parts in section, on a much larger scale, showing the components of FIG. 1 the termination in greater detail;

FIG. 2B is a sectional view taken along line 2B-2B of FIG. 2A;

FIG. 3 is a view similar to FIG. 2A showing a second termination embodiment;

FIG. 4 is a view similar to FIG. 2A showing a third termination embodiment, and

FIG. 5 is a view similar to FIG. 3 illustrating a fourth termination embodiment.

### DETAILED DESCRIPTION OF AN ILLUSTRATIVE EMBODIMENT

Refer now to FIG. 1 which illustrates a skin area of a patient P where catheters C are implanted in the patient by way of a transcutaneous access device A such as the one shown in my above-identified patent. Each catheter C is terminated by a termination assembly shown generally at 10. Each assembly includes a connector 12 and an end cap 14 which may be releasably secured to the connector, the end caps being shown in both unassembled and assembled positions.

In a typical application, the catheters C may be connected to a dialysis machine in order to treat a patient with chronic kidney failure. In that event, with the end caps 14 removed from the respective connectors 12, those connectors are coupled to the mating connectors (not shown) at the ends of tubing leading to and from the dialysis machine. When the connectors 12 are decoupled from their mates, they may be protectively enclosed by end caps 14 as will be described.

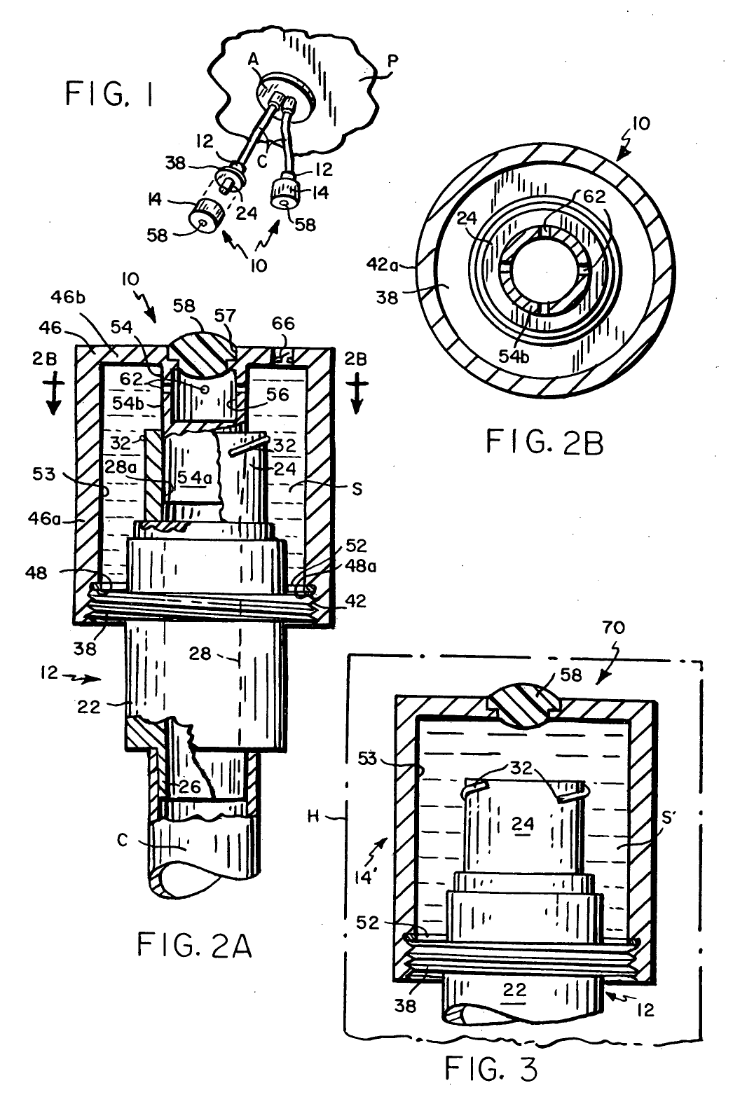

Referring now to FIGS. 2A and 2B, each connector 12 may be a more or less standard connector of the type used to terminate catheters in the medical industry, e.g. a screw-type connector as shown or a luer connector. The illustrated connector 12 attached to each catheter C happens to be a male screw-type connector and includes a tubular main body 22 having a cylindrical coupling stem 24 extending from one end and a nipple 26 extending from the opposite end. An axial lumen 28 extends the entire length of the connector. An end segment of catheter C is engaged over nipple 26 and sealed thereto so that the catheter lumen is in fluid communication with connector lumen 28. The coupling stem 24 has one or more exterior threads 32 of the type commonly found on screw-type connectors of this general type.

The segment 28a of the lumen 28 extending through the stem 24 is slightly tapered as is customary for connectors for this type and is adapted to be coupled to an internally threaded female connector (not shown). Since that female connector is conventional and does not form part of the invention, it will not be described in detail. Suffice it to say that the female connector includes a tube with a cup-like skirt surrounding the tube. A segment of the tube within the skirt is exteriorly tapered and the rim of the skirt is internally threaded so that when the skirt is screwed onto stem 24, the tube is wedged into the lumen segment 28a of stem 24 so that a seal is made between the tube and the stem. The opposite end of that tube is connected to one end of a tube leading to or from an external device, e.g. a dialysis machine.

The only possible difference between connector 12 and a standard connector of the same type is that it has an enlargement 38 between its ends which is larger (in radius) than the stem 24. Furthermore, that enlargement 38 has surfaces at its periphery that can interfit with opposing surfaces on end cap 14. In the illustrated assembly 10, those interfitting surfaces are screw threads 42.

Still referring to FIGS. 2A and 2B, end cap 14 is a cup-like member 46 having a cylindrical side wall or skirt 46a and a discoid end wall 46b. The side wall 46a of cap 14 has an internally threaded counterbore 48 so that the end cap can be screwed onto the threaded flange 38 of the connector 12. The shoulder 48a at the bottom of counterbore 48 may be covered by a soft plastic film 52 to provide a seal when the end cap 14 is screwed onto the connector 12. Thus, when end cap 14 is secured to connector 12, a fluid-tight chamber 53 is formed which completely surrounds connector stem 24.

Of course, other means for releasably connecting cap 14 to connector 12 may be envisioned. For example, flange 38 may be formed with a groove and the side wall of counterbore 48 may have an interior rib which snaps into that groove when cap 14 is seated on flange 38, or vice versa, as will be described later in connection with FIG. 5. Of course with this type of non-screw connection, the enlargement 38 and skirt 46a need not be circular.

As best seen in FIG. 2A, an axial post 54 extends down from the end wall 46b of end cap 14 at the center of that wall. Post 54 has a slight external taper so that when the end cap 14 is screwed onto connector 12, a segment 54a of the post plugs into stem 24 and wedges against the wall of lumen segment 28a providing a fluid-tight seal between the post and stem 24.

While the end segment 54a of post 54 that seats in the lumen segment 28a is solid, the post segment 54b outside the stem 24 is hollow to define a small antechamber 56 between the post segment 54a and the end cap end wall 46b. Also, a small opening 57 is formed in end wall 46b in line with antechamber 56 and that opening is closed by a needle-penetrable septum 58 creating an injection site. Small holes 62 are provided in the side wall of the tubular post segment 54b establishing fluid communication between antechamber 56 and the larger chamber 53 containing connector stem 24.

Thus, using a hypodermic needle (not shown) inserted through septum 58, a liquid sterilizing agent S can be injected into antechamber 56. That liquid will flow through the holes 62 into chamber 53 filling that chamber. Preferably, to facilitate the escape of air from chamber 53 during the filling process, a small check valve 66 may be provided in a wall of end cap 14, e.g., end wall 46b.

It is important to note that while agent S bathes the outside of the catheter stem it cannot flow into stem 24 because of post 54. Thus, patent P is assuredly isolated from the agent S area if a catheter C is elevated so that end cap 14 is uppermost.

During the filing process, the antechamber collects any debris due to penetration of the septum and the bottom wall of the antechamber functions as a needle stop.

In some applications, the antechamber may not be required. In that event, the post 54 may be solid from end to end and the septum 58 located elsewhere on the end cap so that it is not in line with that post. Also, in some applications post 54 may be of a compressible material and simply block the entrance into stem 24 when the end cap 14 is coupled to the connector 12.

In order to use the termination assembly 10, after a particular therapy session has ended, connector 12 may be decoupled from its mating connector leading to the external therapy device, e.g., a dialysis machine. An end cap 14 may then be removed from its sterile package and screwed onto the flange 38 of the connector 12 until a seal is made at the boundary of the end cap and the flange, i.e., at film 52. Then, a sanitizing agent S such as alcohol, bleach or betadyne may be injected through septum 58 into antechamber 56. The injected agent will flow through holes 62 and fill the chamber 53 thereby completely immersing the working end of the connector 12, including its stem 24, in the agent. That portion of the connector may remain immersed in the disinfectant until the next therapy session. At that time, with the end cap pointed downward, the end cap may be unscrewed from connector 12 so that the agent does not spill out of the end cap. The connector 12 may then be immediately coupled to a pre-sanitized mating connector at the end of the catheter leading to the external therapy device. The end cap 14 and its contents may be thrown away or the end cap may be emptied and sent to a sterilization facility so that it can be reused.

Refer now to FIG. 3 which illustrates another termination assembly embodiment, shown generally at 70, which utilizes a biocompatible sanitizing agent S′ such as saline solution or distilled water. Since most of its parts are similar to those of termination 10, those similar parts of termination 70 carry the same identifying numerals.

In this case, the termination assembly includes an end cap 14′ which lacks post 54. This is because the chamber 53 within the end cap 14′ is filled with a biocompatible sterilizing agent S′ which may pass into catheter C without hurting the patient P. The volume of agent S′ inside chamber 53 is then heated by external heating means H to a temperature sufficient to kill any bacteria or microbials on the portion of connector 12 inside that chamber. The external heater H may be a small microwave or RF heater or an electric heater. In any event, the heater heats the volume of liquid inside chamber 53 to a high enough temperature quickly enough to kill the microbials on the connector stem without appreciably heating any saline that may find its way into catheter C. Once sanitized, the contents of the end cap 14′ remain in a sanitary at least until the end cap is removed from the connector 12.

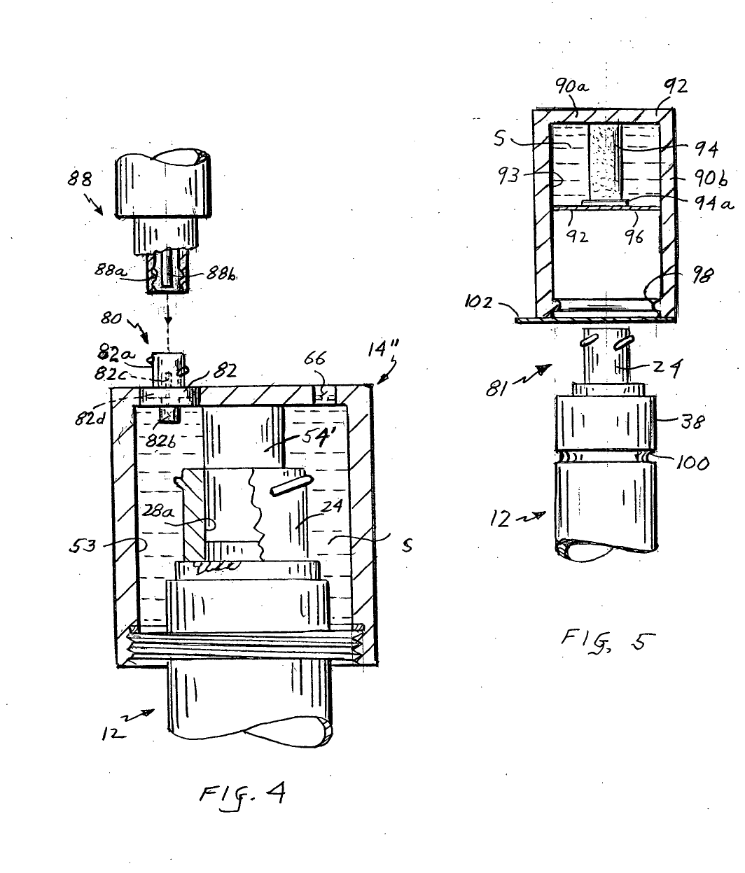

Refer now to FIG. 4 which shows generally at 80 a termination assembly incorporating the invention which is especially adapted to be activated or filled using a so-called needleless hypodermic syringe. Such syringes are becoming quite prevalent in hospitals and clinics for safety reasons, i.e. to eliminate the possibility of hospital personnel being pricked by the needles of conventional hypodermic syringes.

Assembly 80 is similar to assembly 10 depicted in FIG. 2A. It includes an end cap shown generally at 14′ which has a solid post 54′ adapted to plug into connector stem 24 to isolate the lumen segment 28a therein from chamber 53. Also, instead of a septum 58, the injection site in the end cap comprises a valved injection port 82 mounted in the end wall of the end cap 14′ so that the port is offset from post 54′. Injection port 82 is a standard part available from various hospital supply companies. Suffice it to say here that the port has a threaded inlet 82a located outside the end cap and an outlet 82b inside the end cap. A check valve (not shown) inside the port 82 allows fluid flow into the chamber but prevents fluid flow from the chamber. When a needleless hypodermic syringe 88 is coupled to inlet 82a sanitizing agent S may be introduced into the space inside the end cap 14′. In other words, the distal end of the needleless syringe 88 includes an internally threaded sleeve 88a which can be screwed onto the threaded inlet 82a of the injection port 82. When that occurs, a tube 88b inside sleeve 88a engages an actuator 82c inside inlet 82a which unseats a valve member, e.g. a diaphragm 82c, inside port 82 thereby allowing a agent S to flow from the syringe 88 into the chamber. The same valve member preventing fluid constitutes a check valve preventing fluid flow out of the chamber 53.

In this embodiment of the invention as well as the others already described, a vent valve 66 may not always be required because in some applications it may only be necessary to partially, e.g. 75%, fill chamber 53 with agent S; any residual air in the chamber will be compressed somewhat by the infilling agent.

Refer now to FIG. 5 which illustrates yet another connector termination assembly embodiment 82 wherein the sanitizing agent S is contained right in the end cap 90 of the assembly, i.e. the end cap is pre-filled with sterilant. As before, the end cap 90 has an end wall 90a and a side wall or skirt 90b shaped and is shaped and sized to engage a catheter connector 12 having an enlargement 38 which is larger in cross-section than the stem 24 of that connector. End cap 90 is provided with a diaphragm 92 part way along this length which defines with end wall 90a a closed antechamber 93 containing a sanitizing agent S. Extending from the end cap end wall 90a to diaphragm 92 is a resilient, axially compressible post 94, e.g. of a foam material. Preferably, the end of post 94 facing diaphragm 92 has a relatively rigid end 94a. Also, the diaphragm 92 is formed with a tear line or weakness 96 radially out from end 94a so that when the diaphragm is contacted by the free end of stem 24, it will break or tear at line 96.

Also, the side wall or skirt 90b of end cap 90 is formed with a rib or groove 98 near its open end which is adapted to engage or snap into a mating groove or rib 100 formed in the enlargement 38 of connector 12. The aforesaid rib/groove connection is designed so that when end cap 90 is coupled to connector 12 so that there is a rib/groove connection, the entrance into stem 24 will be covered by the diaphragm 92 backed up by the post end 94a before the diaphragm 92 tears at tear line 96 allowing the sterilant S to flow down around, but not into, the stem 24.

In addition to providing a releasable coupling between the end cap 90 and the connector 12, the rib/groove connection 98, 100 also provides a seal between those two members. To optimize the efficacy of that seal, the rib 98 may be of a resilient material.

Other end cap designs comparable to end cap 90 may be envisioned. For example, the closed end segment of end cap 90 may be filled with a porous compressible material, e.g. open cell foam, filled with sterilant S with a center area of that compressible material facing connector 12 being solid so that when the end cap is coupled to the connector, that central portion will cover the entrance to stem 24 after which sterilant will be squeezed by the connector from the compressible material onto the exterior surface of stem 24. In order to prevent evaporation of the sterilizing agent in such an end cap, the open end of the end cap may be closed by a removable impermeable cover of metal foil plastic film or the like as shown at 102 in FIG. 5.

The components of my termination assembly embodiments are preferably molded of a suitable medical grade plastic material able to withstand sterilization and are relative inexpensive to make in quantity. Therefore, the overall cost of the termination assembly should be comparable to that of a conventional male/female connector assembly.

It will thus be seen that the objects set forth above, among those made apparent from the preceding description, are efficiently attained.

Also, since certain changes may be made in the above constructions without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention described herein.

1. A catheter termination assembly comprising
an elongated connector of the type having a coupling stem at one end, a tubing connection nipple at the opposite end and an axial lumen extending, between said stem and said nipple, said connector also including a lateral enlargement between said stem and said nipple, and
a cup-like end cap, said end cap including an end wall, a skirt extending from the end wall and an open end, said skirt being sized to engage around said enlargement, said enlargement and said skirt having interfitting surfaces enabling the end cap to be releasably coupled to the enlargement so as to define therewith a fluid-tight chamber surrounding said stem, and an injection site in a wall of the end cap by which a sanitizing agent may be introduced into said chamber so as to immerse the stem in said agent.

2. The assembly defined in claim 1 and further including a sanitizing agent at least partially filling the chamber.

3. The assembly defined in claim 2 wherein said agent comprises a biocompatible liquid.

4. The assembly defined in claim 3 wherein said agent is saline solution or distilled water.

5. The assembly defined in claim 3 and further including a heater for heating the contents of said chamber to a temperature sufficient to kill microbials on said stem.

6. The assembly defined in claim 2 wherein said agent is a chemical disinfectant, preferably alcohol, bleach, or betadyne.

7. The assembly defined in claim 6 and further including a post extending from the end wall within the end cap and being shaped and sized to block said lumen when the skirt is coupled to said enlargement.

8. The assembly defined in claim 7 wherein said opening and said septum are laterally displaced on the end wall from said post.

9. The assembly defined in claim 7 wherein
the post has a solid segment which plugs into the stem, and a tubular segment outside the stem which defines an antechamber in fluid communication with both said injection site and said chamber.

10. The assembly defined in claim 1 wherein the injection site includes a needle-penetrable septum mounted in said wall of the end cap.

11. The assembly defined in claim 1 wherein the injection site includes a valved injection port mounted in said wall of the end cap.

12. The assembly defined in claim 1 and further including a vent valve mounted in said wall or said skirt for venting the chamber as said agent is being introduced into the chamber.

13. The assembly defined in claim 1 wherein
said enlargement is cylindrical and the interfitting surfaces thereof comprise first threads, and
the skirt is cylindrical and the interfitting surfaces thereof comprise second threads on an interior surface thereof.

14. The assembly defined in claim 1 wherein said interfitting surfaces on said enlargement and said skirt form a rib-in-groove connection.

15. A catheter termination assembly comprising
a catheter connector of the type having a coupling stem at one end, a catheter connection nipple at the opposite end and an axial lumen extending between said stem and said nipple, said connector also including a circular enlargement between said stem and said nipple, said enlargement having a threaded circumferential surface, and
a cup-like end cap including an end wall and a cylindrical skirt extending from the end wall, said skirt having an interiorly threaded end segment sized to threadedly engage around said enlargement so that the end cap may be screwed to the enlargement to form therewith a fluid-tight chamber encircling said stem, and a fluid injection site in said end wall by which a sanitizing agent may be introduced into said chamber so that said stem is immersed in said agent.

16. The assembly defined in claim 15 and further including a sanitizing agent in said chamber.

17. The assembly defined in claim 15 and further including a vent valve mounted in the wall or skirt of said end cap for venting said chamber as said agent is being introduced into said chamber.

18. The assembly defined in claim 15 wherein the threaded end segment of the skirt has a larger inside diameter then the remainder of the skirt and defines a flat sealing surface for the end cap.

19. The assembly defined in claim 15 and further including an axial post extending from the end wall into the end cap and being shaped and sized to block said lumen when the end cap is screwed onto said enlargement.

20. The assembly defined in claim 19 wherein said injection site is radially displaced on the end wall from said post.

21. The assembly defined in claim 19 wherein
the post has a solid segment which plugs into the stem, and
a tubular segment outside the stem which defines an antechamber in fluid communication with both said injection site and said chamber.

22. The assembly defined in claim 19 and further including a chemical sanitizing agent filling said chamber.

23. The assembly defined in claim 22 wherein said agent is selected from the group consisting of alcohol, bleach and betadyne.

24. The assembly defined in claim 16 wherein the sterilizing agent is a biocompatible liquid.

25. The assembly defined in claim 24 and further including a heater sized to receive the screwed-together connector and end cap for heating the contents thereof to a temperature sufficient to kill organic contaminants on said stem.

26. The assembly defined in claim 15 wherein said connector and said end cap are of a medical grade plastic material able to withstand sterilization.

27. The assembly defined in claim 15 wherein the injection site includes a needlepenetrable septum mounted in said wall of the end cap.

28. The assembly defined in claim 15 wherein the injection site includes a valved injection port mounted in said wall of the end cap.

29. A catheter termination assembly comprising
an elongated connector of the type having a coupling stem at one end, a tubing connection nipple at the opposite end and an axial lumen extending between said stem and said nipple, said connector also including a lateral enlargement between said stem and said nipple, and
a cup-like end cap, said end cap including an end wall and a side wall extending from the end wall, said side wall defining an end opening and being sized to engage around said enlargement, said enlargement and said side wall having interfitting surfaces enabling the end cap to be releasably coupled to the enlargement so as to define therewith a fluid-tight chamber surrounds a portion of which said stem, means for providing a sanitizing agent in the end cap so that when the end cap is coupled to the enlargement, said agent may be introduced into said chamber portion, and means in the end cap for engaging and end of said coupling stem and blocking said lumen.

30. The assembly defined in claim 29 wherein the said providing means comprise an injection site in a wall of said end cap.

31. The assembly defined in claim 29 wherein the sterilant providing means comprise an interior diaphragm extending from the side wall of the end cap and being spaced from said end wall to define an agent-containing antechamber within the end cap, said diaphragm adapted to be pierced by said stem when the end cap is coupled to said enlargement.

32. The assembly defined in claim 29 wherein the blocking means comprise a post extending from said end wall to said stem, said end post being dimensioned and adapted to block said lumen when said end cap is coupled to the enlargement.

33. The assembly defined in claim 32 wherein the post is axially compressible and resilient.

34. The assembly defined in claim 29 wherein
said providing means comprise a body of compressible, porous, disinfectantimpregnated material partially filling the end cap adjacent to said end wall, and
said blocking means comprise a relatively rigid diaphragm portion disposed opposite said end of the stem.

35. A method of sanitizing a connector of the type having a coupling stem at one end, a tubing connection nipple at the opposite end, an axial lumen extending between the stem and the nipple and a lateral enlargement between the stem and the nipple, said method comprising the steps of
providing an end cap having a closed end and an open end sized to engage around said enlargement so as to define with said enlargement a fluid-type chamber surrounding said stem:
introducing a sanitizing agent into the portion of said chamber occupied by said stem, and
blocking said lumen so as to prevent said agent from entering said connector.

36. The method defined in claim 35 wherein said agent is introduced by injecting it into said chamber.

37. The method defined in claim 35 wherein said agent is provided by releasing it from an agent-filled antechamber formed in the end cap adjacent to a closed end thereof and outside of said chamber portion.
